(19) United States
(12) Patent Application Publication
(10) Pub. No.: US 2026/0287585 A1
(43) Pub. Date: Sep. 24, 2026

(54) A METHOD FOR OPTICAL, LABEL-FREE DETECTION OF MOLECULES AT ULTRA-LOW CONCENTRATION, PARTICULARLY AS LOW AS ZEPTOMOLAR CONCENTRATION

(71) Applicant: Università degli Studi di Bari Aldo Moro, Bari (IT)

(72) Inventors: Luisa TORSI, Bari (IT); Eleonora MACCHIA, Bari (IT); Gaetano SCAMARCIO, Bari (IT); Cinzia DI FRANCO, Bari (IT)

(21) Appl. No.: 19/472,313

(22) PCT Filed: Apr. 6, 2023
(86) PCT No.: PCT/IB2023/053542

§ 371 Date: Oct. 3, 2025

(51) Int. Cl. G01N 33/543; G01N 21/59; G01N 33/553

(52) U.S. Cl. CPC G01N 33/54393; G01N 21/59; G01N 33/553; G01N 2021/5903

(57) ABSTRACT

Detecting a binding event of specific binding pair-forming substances, comprising includes: providing a layer of a first specific binding pair-forming substance (SBPS) on a substrate, providing an enclosure of the layer with a first dielectric medium (DM) rinsing the enclosure for a first time period with a second DM, restoring the first DM in the enclosure following the rinsing, feeding a solution of a second SBPS to the enclosure to cause an interaction between the first second SBPS following the restoring, incubating the enclosure for a second time period, the interaction including a binding event when the first and second SBPS provide a specific binding pair, detecting a shift in a parameter representative of a layer dielectric function following the incubating, comparing the shift with a threshold, whereby when the shift exceeds the threshold the binding event has occurred.

### TECHNICAL FIELD

The present invention relates to optical, label-free assay of molecules at extremely low concentration, particularly at concentrations as low as 10 zeptomolar concentrations.

### PRIOR ART

Near-field approaches involving nanometer-size interfaces can enable label-free detections at single-molecule resolution.¹ Namely, a transducing nano-interface, biofunctionalized with few recognition elements (e.g., antibodies) can detect a train of single antigen-antibody affinity binding events. The occurrence of such events is, however, limited by the antigen Brownian diffusion²⁻⁴ which makes its bumping into the nanometric interface, aleatory. Under these conditions, binding can occur in a few minutes only when the capturing nano-interface and an antigen are restrained to a femtoliter volume. This means that in a 0.1 ml volume to be analyzed, at least 10¹¹ antigens (nM-10⁻⁹ mole/liter) are needed. Hence, a near-field approach actuated with a nanometric interface involves the study of a sequence of events at the single-molecule resolution but does not enable assaying a single-molecule in 0.1 milliliter or, equivalently, at the single-molecule limit-of-detection (LOD) or limit-of-identification (LOI) in 0.1 ml volume, reaching LODs of 10⁻²⁰ mole/l (M).

Optical methods for single-molecule investigations are no exception as they heavily rely on near-field approaches where femto/picomolar (10⁻¹⁵/10⁻¹² M) detection limits are achieved at most.⁵⁻⁹ This low, but still far from single-molecule LODs, are reached by adopting strategies exploiting effects such as the localized surface plasmon resonance⁷ or the plasmon-enhanced Raman spectroscopy.⁹ Standard Surface Plasmon Resonance (SPR)¹⁰⁻¹² LODs for antigens captured by a layer of antibodies deposited on the gold SPR slide are, notoriously, in the 10⁻⁸ M regime.¹³⁻¹⁸

Lately, label-free biochemical sensing at the single-molecule LOD in 0.1 ml has been demonstrated by means of bioelectronic devices endowed with a large (millimeter wide) detecting interface,^{2,19-22} as well as with a Kelvin probe force microscopy (KPFM)²³ investigation exploring a 10⁴ μm² wide interface. In both cases the surface is packed with a 10⁴/μm² density of capturing elements. The KPFM study shows how an extended surface potential shift upon affinity binding of 10±3 antigens, is measured directly on a specimen's surface populated by 10⁸ capturing antibodies. The key to overcoming the diffusion limit, plaguing near-field approaches, has been proven to be the millimeter-wide capturing surface, itself. Such a large detecting interface, enormously enhances the probability of a few antigens in 0.1 ml, to be captured by one of the 10¹¹-10¹² of antibodies attached to it.²⁴

The elicited large-area bioelectronic approaches both involve an applied electric field, which has been deemed necessary to enable the detection of the single-binding event with a sufficiently high signal-to-noise ratio. To this end, an electrostatic domino-like amplification process is in fact postulated to enable the propagation of the electrostatic conformational change of the antibody involved in the single-antigen binding event. The electrostatic change starts from this antibody, and propagates, supposedly fueled by the applied electric field, to the neighboring antibodies, changing in turn their conformational state. The propagation advances until a large portion of the trillion antibodies populating the millimeter-wide gate electrode, have been switched.^{19,20} The detailed mechanisms of such an amplification process, necessary to make the insignificant single-binding electrostatic change detectable, remains elusive.

### OBJECT OF THE INVENTION

The object of the invention is to solve the technical problems noted in the prior art. Specifically, it is an object of the invention that of providing a method to assay molecules at ultra-low concentration, particularly at single-molecule level in 0.1 ml (10⁻²⁰ M) with an optical label-free approach.

### SUMMARY OF THE INVENTION

The object of the invention is achieved by a method having the features of the claims that follow, which form an integral part of the technical disclosure provided herein in relation to the invention. Specifically, the optical, label-free approach of the method of the invention is carried out through Surface Plasmon Resonance (SPR).

### BRIEF DESCRIPTION OF THE FIGURES

The invention will now be disclosed with reference to the attached figures, provided purely by way of non-limiting examples, and wherein:

FIG. 1(a) is a schematic representation of Surface Plasmon Resonance (SPR) layers and of the surface-plasmon mode propagating at the interface between a metal and a dielectric layer,

FIG. 1(b) represents a structure of a SPR apparatus in the Kretschmann configuration,

FIG. 1(c) represents SPR angular reflectivity curves measured with the 670 nm laser source at a gold surface (hollow squares) and at the physisorbed anti-IgG layer (hollow circles) in PBS, together with fitting curves;

FIG. 1(d) represents SPR trace (plasmon peak angle shift vs. time) during the physisorption of the anti-IgG layer on the SPR gold slide;

FIG. 2(a) represents an IgG SPR assay using a physisorbed anti-IgG capturing layer comprising (2.8±0.4)·10¹¹ antibodies;

FIG. 2(b) represents an IgG calibration curve (hollow squares) and negative control experiment with a bovine serum albumin protein as non-capturing layer (hollow circles); the LOD is estimated as the average of the noise in the negative control experiment plus three times its standard deviation³⁰;

FIG. 3(a) illustrates an SPR transient signal recorded according to Protocol A disclosed in the following;

FIG. 3(b) is an enlarged view of a portion of FIG. 3(a) identified by a dotted line frame, and encompassing only steps 1-7;

FIG. 4(a) illustrates an SPR transient signal recorded according to Protocol B disclosed in the following;

FIG. 4(b) is an enlarged view of a portion of FIG. 4(a) identified by a dotted line frame, and encompassing only steps 1-7,

FIG. 5(a): SPR transient signal recorded according to Protocol C disclosed in the following;

FIG. 5(b): SPR transient signal recorded according to Protocol D disclosed in the following;

FIG. 6(a): SPR transient signal recorded according to Protocol E (disclosed in the following) on an anti-IgG film physisorbed on the gold-coated slide;

FIG. 6(b): SPR transient signal recorded according to Protocol E (disclosed in the following) on an anti-IgG physisorbed on gold nanoparticles-based film deposited on the gold-coated slide.

FIG. 7: SPR transient signal recorded according to Protocol F (disclosed in the following) on an anti-IgG physisorbed on gold nanoparticles-based film deposited on the gold-coated slide.

FIG. 8: SPR θ shifts as compared to a baseline, given in Table 2, for all protocols A-F at IgG 100 zM, 1 aM, and 100 nM. The results from a further protocol, referred to as “protocol F” are also added. The negative control experiment is carried out with IgM at 1 fM. All the experiments are carried out on an anti-IgG physisorbed capturing layer on the gold-coated slide.

FIG. 9: is a graphical representation of a 2² factorial design of protocols A-D.

FIG. 10(a): graphical representation of coefficients of a model of SPR angle shift at 100 zM of the 2² factorial design of FIG. 9.

FIG. 10(b) shows an isoresponse contour plot of the SPR angle shift in the whole experimental domain response at 100 zM of the 2² factorial design of FIG. 9,

FIG. 10(c) shows a graphical representation of the coefficients of the model of the SPR angle shift at 100 aM of the 2² factorial design of FIG. 9,

FIG. 10(d) shows an isoresponse contour plot of the SPR angle shift in the whole experimental domain at 100 aM response of the 2² factorial design of FIG. 9,

FIGS. 10(e) and 10(f) are graphical representations, respectively, of the coefficients of the model of the SPR angle shift at 100 nM and upon exposure to IgM 1 fM (negative control experiment) of 2² factorial design. The error bars are the confidence level at p=0.05, while the stars indicate the significance of the coefficients (*=p<0.05, **=p<0.01, ***=p<0.001).

FIG. 11(a) shows a graphical representation of an assay carried out in physiological conditions showing how a real marker such the HIV-1 p24 (10 zM and 100 zM) can be detected in a physiological, buffer at the single molecule level in 0.1 ml, in the presence of an interferent protein such as MERS-CoV spike S1 (1 fM). “Protocol D” is used in this case.

FIG. 11(b) the same as FIG. 11(a) but, to better simulate the assay on a patient fluid only the baseline in the reference fluid (HEPES buffer 1, iₛ=150 mM and pH=7.4) followed by rinsing with HEPES buffer 4 (iₛ=150 mM and pH=6), and the assay carried out in a solution containing both the MERS-CoV spike S1 (1 fM) interferent protein and the marker to be detected, HIV-1 p24, at 10 zM, is proposed.

FIG. 11(c) the same as panel (b) but the rinsing, with HEPES buffer 4 (iₛ=150 mM and pH=6), is performed only after the sensing (carried out in a solution containing both the MERS-CoV spike S1, 1 fM, interferent protein and the marker to be detected, HIV-1 p24) at 10 zM is proposed.

By way of preliminary remark, the figures attached to the disclosure may contain, in addition to reference numbers or letters, text matter with the purpose of enhancing the understanding of the very figures, especially in view of the nature of the invention. The text matter included in the figures may be repeated in the following disclosure as well.

### DETAILED DESCRIPTION

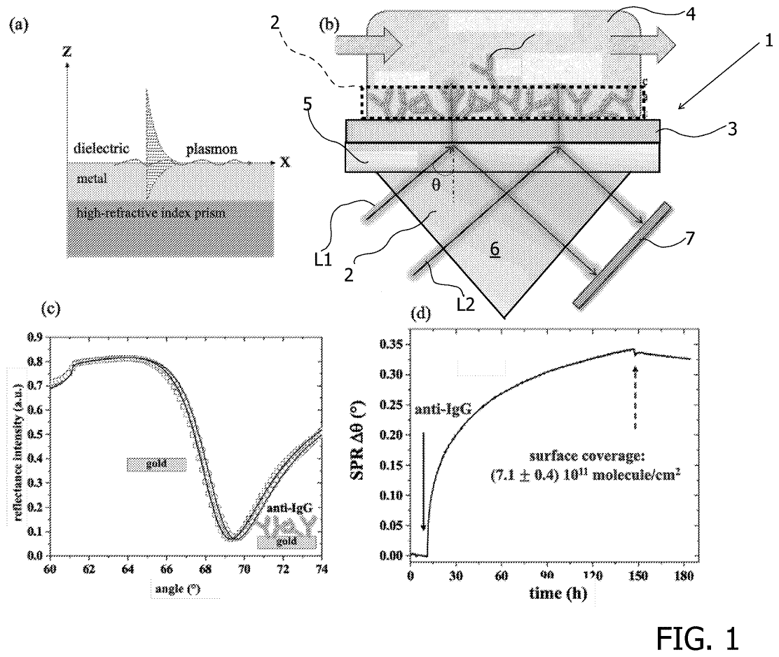

Surface Plasmon Resonance (SPR) relies on the evanescent wave associated with the total internal reflection of a laser at the boundary between a higher refractive index prism and a dielectric medium, as also shown in FIG. 1(a)¹². The impinging light will be totally (internally) reflected if the angle of incidence exceeds a critical value (Bruster angle).

Surface plasmons arise at an interface between a metal endowed with a dielectric function (real part) ε′ₘ<0 and a dielectric medium (e.g., water) with ε′_{d}>0.¹⁵ The intense optical field associated with the collective oscillation propagates along the metal-dielectric interface (x-axis in FIG. 1(a)) and decays exponentially in both the metallic and dielectric media. Such an evanescent character gives surface specificity to the technique and allows the monitoring of a 30-300 nm layer deposited at the metal/liquid interface.

SPR does not envisage an applied electric field but rather an optical field where charge oscillations (plasmons) at a metal/dielectric interface are optically excited.^{15,12}

More specifically, the invention comprises a new method involving a change the pH of the solution that rinses the detecting interface, as compared to the physiological solution (iₛ=150 mM and pH=7.4) in which the single-molecule affinity binding is carried out. This by itself, enables the optical label-free detection at a LOI of 10⁻²⁰ M. A change and the ionic strength (is) alone does not generate the elicited effect although, along with a pH change, can contribute to enhance the signal. The rinsing preferably is performed before the sensing.

With reference to FIG. 1(b), reference number 1 designates an assay setup (as well as an experimental setup) configured for carrying out a method according to the invention. All the experiments referred to herein have been performed with a BioNavis-200 Multi-parameter Surface plasmon resonance (MP-SPR) Navi™ instrument, in the angle modulation Kretschmann configuration of FIG. 1 (b). Here, the evanescent wave monitors a capturing anti-Immunoglobulin G (anti-IgG) layer 2 (in general, according to the invention, a layer of a first specific binding pair forming substance) deposited on a gold substrate 3. The SPR modulus is equipped with two laser sources L1, L2 (both at λ=670 nm) impinging onto 3 mm apart areas of the specimens, to assess for its uniformity. The two measured traces were almost identical in all the experiments referred to herein, proving that the homogeneity of the capturing antibody layer properties was always extremely high.

Hence, for the sake of clarity, only the trace coming from one laser source is shown in the experiments here presented. The laser beam incident angle, θ (larger than the total reflection critical angle) was varied in the 50.290°-77.930° range with an instrumental error of 0.002°. A flowthrough cell 4 with an internal volume of 0.1 ml served as fluidic. The injections into the cell were performed manually with a 1 ml sterile syringe and the experiments were carried out at room temperature. All the proteins used in the experiments referred to herein (anti-IgG, IgG, IgM) are polyclonal antibodies from Sigma-Aldrich and used with no further purification.

The sensor slide (SPR Navi-200), allocated in the sample holder, comprised an optical glass 5 matching the high refractive index of a prism 6, covered by a semi-transparent film of thermally evaporated gold (~50 nm) deposited on a chromium adhesion layer (~2 nm). Before use, the slides were dip cleaned in an NH₄OH/H₂O₂ aqueous solution (1:1:5 v/v) at 80-90° C. for 10 min; rinsed with water afterward, dried with nitrogen, and treated for 10 min in a UV-ozone cleaner. Heading 7 indicates a photodetector configured to detect reflected beams from L1 and L2.

### Results

The capturing anti-IgG layer 2 was deposited by injecting 0.1 ml of a 50 μg/ml anti-IgG phosphate buffer saline (PBS, is =163 mM and pH=7.4) solution in the flow cell 2. The SPR slide (glass 5), previously allocated in the cell 2, was left in contact with the anti-IgG solution for 2 hours, while measuring the SPR signal.

In FIG. 1(c) the angular reflectivity curves encompassing the plasmonic peak as a function of the incidence angle θ is shown for the bare sensor slide (hollow squares) and for the slide covered by the physisorbed anti-IgG layer (hallow circles).

The experimental curves were simulated with a multilayer model based on the Fresnel equation using Winspall 3.02 software.²⁶ The thicknesses and optical parameters returned from the simulation of the SPR curves are summarized in the following Table 1.

| | | Refractive | Extinction |
|---|---|---|---|
| Layer | Thickness/nm | Index | Coefficient |
| Glass BK7 | — | 1.518 | 0 |
| Cr | 2.0 ± 0.1 | 3.3 ± 0.2 | 2.8 ± 0.4 |
| Au | 41 ± 1 | 0.16 ± 0.02 | 3.85 ± 0.05 |
| anti-IgG | 6.9 ± 0.5 | 1.374 ± 0.00 | 0 |
| PBS | — | 1.330 | 0 |

Those obtained for the bare Au-coated slides are in perfect agreement with the nominal thicknesses declared by the SPR slides' provider and with the relevant optical parameters given in the literature,²⁷ proving the reliability of the simulation performed. The resulting value for the simulated thickness of the anti-IgG layer is 6.9±0.5 nm, where the error bar was estimated as the relative standard deviation on the two different sampled areas.

In FIG. 1(d) the SPR plasmon peak angle shift vs. time is recorded as the anti-IgG layer 2 adsorbs on the slide. The surface coverage of anti-IgG physisorbed on the gold surface 3 was quantitatively assessed, as customary in SPR experiments, by means of the de Feijter's equation^{28,21} resulting in a surface coverage of (7.1±0.4)·10¹¹/cm². In general, according to the invention, the layer 2 of a first specific binding pair forming substance has a density—or surface coverage—between 10² and 10⁴ per μm² of the substrate 3 on which the layer 2 is provided (a gold surface in the setup of FIG. 1(d)).

An immunoglobulin antibody holds a y-shaped structure of 14.5 nm×8.5 nm×4.0 nm,²⁹ hence 6.9±0.5 nm thickness suggests that a single monolayer encompassing (2.8±0.4)·10¹¹ antibodies is deposited on the 0.4 cm² portion of the gold slides. The theoretical coverage of one layer of antibodies (standing or laying down) corresponds approximately to 10¹² molecules/cm².²¹ These data concur to support a model of a single layer of highly packed anti-IgG proteins physisorbed in a non-ordered fashion, covering the gold surface.

A second capturing layer was produced according to the following procedure. A gold nanoparticle (Au NP) suspension at a concentration of 3.45-10⁹ particles/ml in PBS 0.1 mM, was purchased from Sigma-Aldrich (Prod. Num.753688) and used as received, with no further purification. The nanoparticles hold a diameter of 100 nm and were already stable with no need for a capping agent. At first, for the surface modification of Au NPs with anti-IgG antibodies, 4 ml of the nanoparticles' suspension was mixed with 180 μl of an anti-IgG solution (0.1 mg/ml) in PBS.

Thus, the solution contains a total number of nanoparticles of about 1·10¹⁰ nanoparticles mixed with 1·10¹⁵ antibodies. This is meant to have an excess of anti-IgG to fully cover the surface of the NPs. Indeed, a single 100 nm-sized NPs can host about 540 anti-IgGs. The solution of the NPs and the anti-IgGs was left under stirring at 525 rpm for 1 hour at 20-22° C., to accomplish the antibodies' physisorption. Then, the solution was centrifugated at 8000×g for 30 minutes and the supernatant, containing the excess of non-physisorbed anti-IgGs, was removed. The remaining pellet was re-suspended in 1 ml of fresh PBS to have a final concentration of modified NPs (anti-IgG NPs) of about 1·10¹⁰ nanoparticles/ml. The colloidal solution was stirred with Vortex (1000 rpm) for 1 minute and sonicated in an ultrasonic bath for 20 minutes. The solution thus obtained was used for the SPR gold slide modification. To this end, the anti-IgG NPs solution was injected into an SPR flow cell in contact with a bare gold SPR slide. Five injections of 500 μL were repeated by flowing the same solution through the flow cell. During each injection the solution of biofunctionalized NPs was left in contact with the SPR slide for 15 minutes, to fully cover the sensor area (0.42 cm²). The SPR slide prepared according to the protocol can host about 5·10⁹ anti-IgG NPs.

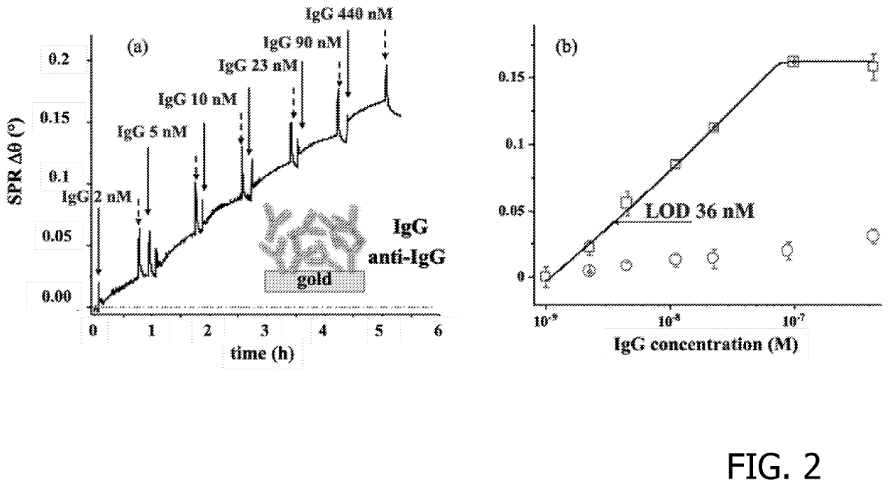

The capturing of the IgG antigens on the physisorbed capturing antibody on gold is then studied measuring the Δθ shift as a function of time. The data are given in FIG. 2(a) where a typical SPR assay of IgG antigens (PBS solution in the 2 nM-440 nM range) incubated with an anti-IgG capturing layer, is shown.

In FIG. 2 (b) the relevant calibration curve is shown (squares) along with the negative control experiment (circles) where a non-capturing bovine serum albumin (BSA) physisorbed layer (BSA 0.1 mg/ml for 2 hours) is entailed. The latter allows for estimating the noise level and its standard deviation. The LOD (Limit-Of-Detection) level, estimated as the negative control experiment average signal plus three times its standard deviation,³⁰ corresponds to a concentration of 36 nM, in line with literature data.

Moreover, it is clear from the minimum concentration needed to detect a significant signal, that the change in SPR signal (associated with the change in dielectric function at the metal-water interface) is due to the formation of a staking layer of captured IgG antigens on the anti-IgG capturing film 2. The IgG layer at the saturation of the SPR signal comprises (1.4±0.8)·10¹¹ (on the 0.42 cm² slide surface) antigens, meaning that there is one dense layer of IgGs, approximately one every two anti-IgGs. The simulated thickness²¹ of the IgG layer (on top of the 6.9±0.5 nm thick anti-IgG) is 1.2±0.8 nm. Such a small thickness should be ascribed to a lay-down orientation of the IgG antigens on the anti-IgG layer 2, which is also in line with previously published studies.³¹

With the aim of investigating the SPR sensing signal at very low LODs, experimental protocols A-F (B-F corresponding to embodiments of the method of the invention) have been set up that will be disclosed in the following.

The negative control experiments involve the measurement of the SPR trace (plasmon peak angle shift Δθ vs. time) at the anti-IgG covered slide upon injection of the non-affinity-binding IgM antigen at 1 fM (10⁻¹⁵ M). The level of the noise is computed considering all the negative control experiments (performed according to the different protocols, vide infra) with their average Δθ shift 0.003° and standard deviation 0.002°. The angle shift is always evaluated after the exposure step to IgM 1 fM in buffer lasting 45 minutes and the rinsing step in buffer or water lasting 20 minutes. The Limit-Of-Detection (LOD) level is computed as the average noise plus three times its standard deviation³⁰: LOD-level=0.009°. The Limit-Of-Identification (LOI) level is the average noise plus six times its standard deviation:³⁰ LOI-level=0.015°. At the LOI level, the detection is very reliable as both false-negative and false-positive random errors are below 1%. This leads to diagnostic sensitivity, specificity, and selectivity of at least 99%.

After the evaluation of the level of the negative control experiment, the trace measurement continues with the injection of the IgG at progressively higher concentration starts, spanning the 10 zeptomolar (10⁻²¹ M, zM)-100 nM concentration range. Each incubation with the affinity binding IgG solution, lasts 45 minutes and it is followed by a rinsing step for 20 minutes.

In a first experimental protocol, referred to herein as “Protocol A”, a 4-(2-hydroxyethyl)-1-piperazineethanesulfonic acid (HEPES) buffer comprising the zwitterionic HEPES molecule, and sodium chloride with is =150 mM, pH=7.4 was chosen to mimic physiological conditions and will be addressed as “HEPES buffer 1”. Considering that the majority of antibodies hold a more basis isoelectric point (pI>6 and for anti-Ig in the 6.4-7.6 range)^{32,33} at physiological conditions the anti-IgG layer should be slightly negatively charged.

The slide, covered with the anti-IgG layer 2 physisorbed from PBS, is allocated in the SPR flow cell 4. At each step of all the experiments the assay solution offering physiological conditions, HEPES buffer 1 comprising either IgM (non-binding antigen serving for the negative control experiment) or IgG (affinity binding species to be detected), is injected (0.1 ml) into the cell and left to incubate in contact with the capturing layer, for 45 minutes. When IgG is present the binding actually occurs. Then the capturing layer is washed with 3 ml of the HEPES buffer 1 comprising no antigen. This step lasts for 20 minutes. More specifically, the experiment begins with the injection of 0.1 ml IgM 1 fM in the HEPES buffer 1 which is incubated for 45 minutes. The sensor surface is then rinsed injecting 3 ml of bare HEPES buffer 1 and it is left to rest for 20 minutes. It proceeds with the injection of IgG solutions still in the HEPES buffer 1 (0.1 ml) spanning the 100 zM-100 nM concentration range. Each incubation step, in which IgG binds to the capturing anti-IgG layer, is followed by a rinsing step.

More schematically, the first experimental protocol, referred to in the following as “Protocol A” comprises:
0. Anti-IgG physisorption from PBS on the slide (2 hours);
1. Baseline (45 min): injection of HEPES buffer 1 (iₛ=150 mM, pH=7.4);
2. Rinsing (20 min): injection of HEPES buffer 1;
3. Negative control experiment (45 min): incubation of IgM 1 fM in HEPES buffer 1;
4. Rinsing (20 min): injection of HEPES buffer 1;
5. Sensing at 100 zM (45 min): incubation of IgG 100 zM in HEPES buffer 1;
6. Rinsing (20 min): injection of HEPES buffer 1;
7. Sensing at 1 aM (45 min): incubation of IgG 1 aM in HEPES buffer 1;
8. Rinsing (20 min): injection of HEPES buffer 1;
9. Sensing at 100 aM (45 min): incubation of IgG 100 aM in HEPES buffer 1;
10. Rinsing (20 min): injection of HEPES buffer 1;
11. Sensing at 100 fM (45 min): incubation of IgG 100 fM in HEPES buffer 1;
12. Rinsing (20 min): injection of the HEPES buffer 1;
13. Sensing at 100 fM (45 min): incubation with IgG 100 zM in HEPES buffer 1;
14. Rinsing (20 min): injection of the HEPES buffer 1.

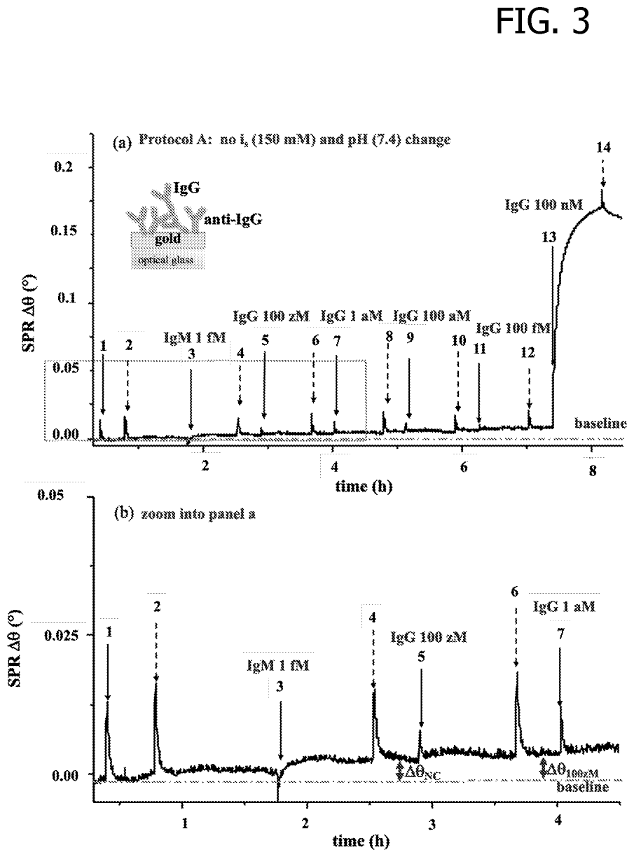

The SPR angle shifts (Δθ) vs. time of Protocol A are shown in FIG. 3. Each step of the protocol is marked by the relevant number from the above list and the baseline is the SPR signal level in HEPES buffer 1 of the stabilized pristine anti-IgG layer.

In FIG. 3(a) the whole sequence of steps 1-14 in the list above is shown while in FIG. 3(b) an enlarged view of steps 1-7 is provided. During each injection, a spike in the SPR signal (variation of the angle θ, that is, Δθ) is seen that in about 2-3 minutes returns to the baseline. As it is apparent and somehow expected FIG. 3(a) shows how no appreciable shift from the baseline until the 100 nM IgG solution is injected. A closer inspection in the below-fM range (FIG. 3(b)) shows a negligible SPR angle shift of Δθ_{100zM}=0.005°±0.0020 (average over 4 replicates) after step 6, namely after the 100 zM IgG injection and rinsing. This is even more evident if compared to the signal level of the negative control (NC) experiment after washing (step 4) which is Δθ_{NC}=0.0020±0.002°. Indeed, Δθ_{100zM}−Δθ_{NC}=0.003°±0.003° of the order of the instrumental error, where the error has been computed as the propagation error.

However, no appreciable further change is seen after step 6 as shown by the relevant arrow. This means that no significant change occurs when 100 zM IgG is injected in the cell as compared to the negative control experiment signal level. Overall, the data show that not enough antigen molecules are provided in the solution to build a layer of IgG on top of the capturing anti-IgG until a 100 nM IgG solution is injected. Only at this IgG concentration, an appreciable change in the dielectric function is revealed by the SPR evanescent wave, which is related to the deposit of an IgG layer on top of the anti-IgG capturing one—layer 2.

A second experiment, referred to as “Protocol B”, comprises also incubation steps carried out in 0.1 ml of HEPES buffer 1 offering the physiological conditions suitable for the IgG binding to occur. These steps last 45 minutes. The washing is carried out in a solution referred to as “HEPES buffer 2” (iₛ=5 mM, pH=6) where both the pH and the ionic strength are lowered. This protocol involves the injection of 3 ml of solutions (HEPES buffer 1 and HEPES buffer 2) characterized by a different salinity. This results in a change in the dielectric function measured in the cell that is ascribable to both the solution and to the capturing layer. To measure the Δθ shift associated with changes in the sole capturing layer upon the washing, a step is introduced and addressed as “restoring of the physiological conditions”. Hence, Protocol B starts with the injection of 0.1 ml the HEPES buffer 1 (either with IgM or IgG) incubated for 45 minutes. Then HEPES buffer 2 (with no antigen dissolved) is injected and left for 20 minutes to wash the capturing layer. Here a large shift of the Δθ angle is measured. Afterward, a new batch of 0.1 ml of HEPES buffer 1 is injected and left for 5 minutes to restore physiological conditions, along with the Δθ angle shift associated with the change of the solution. More schematically Protocol B is carried out according to the following steps:
0. Anti-IgG physisorption from PBS on a 0.4 cm² portion of the slide (2 hours);
1. Baseline (45 min): injection of HEPES buffer 1 (iₛ=150 mM, pH=7.4);
2. a) Rinsing (20 min): injection of HEPES buffer 2 (is =5 mM, pH=6);
   b) Restoring physiological conditions (5 min):
injection of the HEPES buffer 1;
3. Negative control experiment (45 min): incubation with IgM 1 fM in HEPES buffer 1;
4. a) Rinsing (20 min): injection of HEPES buffer 2;
   b) Restoring physiological conditions (5 min): injection of the HEPES buffer 1;
5. Sensing at 100 zM (45 min): incubation of IgG 100 zM in HEPES buffer 1;
6. a) Rinsing (20 min): injection of HEPES buffer 2;
   b) Restoring physiological conditions (5 min): injection of the HEPES buffer 1;
7. Sensing at 1 aM (45 min) incubation of IgG 1 aM in HEPES buffer 1;
8. a) Rinsing (20 min): injection of HEPES buffer 2;
   b) Restoring physiological conditions (5 min): injection of the HEPES buffer 1;
9. Sensing at 100 aM (45 min): incubation of IgG 100 aM in HEPES buffer 1;
10. a) Rinsing (20 min): injection of the HEPES buffer 2;
   b) Restoring physiological conditions (5 min): injection of the HEPES buffer 1;
11. Sensing at 100 fM (45 min): incubation of IgG 100 fM in HEPES buffer 1;
12. a) Rinsing (20 min): injection of the HEPES buffer 2;
   b) Restoring physiological conditions (5 min): injection of the HEPES buffer 1;
13. Sensing at 100 nM (45 min): incubation with IgG 100 zM in HEPES buffer 1.
14. a) Rinsing (20 min): injection of the HEPES buffer 2;
   b) Restoring physiological conditions (5 min): injection of the HEPES buffer 1.

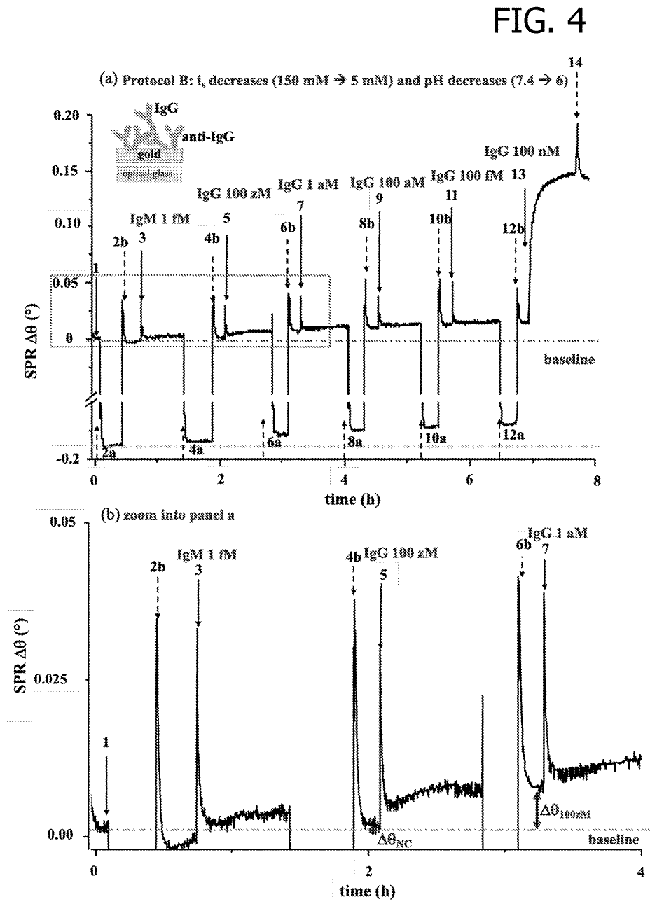

The data relevant to Protocol B are provided in FIG. 4. Notably, while in Protocol A both sensing (affinity binding) and rinsing are carried out in the same HEPES buffer 1 environment, in Protocol B the sensing is still carried out in HEPES buffer 1, but the rinsing involves HEPES buffer 2 entailing a decrease in both pH and iₛ.

As it is apparent in FIG. 4(a) and FIG. 4(b), a negligible shift from the baseline (SPR signal of the pristine anti-IgG layer in HEPES buffer 1) is seen for the negative control experiment after the rising in HEPES buffer 2 and physiological conditions restoring in HEPES buffer 1 (step 4(b)) as Δθ_{NC}=0.002°±0.002°. On the contrary, it is seen that a sizable shift from the baseline is seen upon incubating the anti-IgG layer into a 100 zM IgG solution. After rinsing and restoring of the physiological conditions (step 6(b)) a Δθ_{100zM}=0.019°±0.002° is measured resulting in a Δθ_{100zM}−Δθ_{NC}=0.017°±0.002°. In 0.1 ml at 100 zM only 10±3 IgG molecules are present which can bind only a few out of the 10¹¹ anti-IgG present in the capturing layer. Hence the change in the dielectric function at the interface with the gold layer 3 involves a change occurring in the anti-IgG layer. The signal shift slightly increases when the anti-IgG is incubated with solutions at higher IgG concentrations up to 100 fM. At 100 nM, a much larger SPR Δθ shift is recorded. Here a new layer of IgGs binding to anti-IgG forms, and the following can be stated:
i) the rinsing with HEPES buffer 2 creates a regime of dielectric changes involving the sole anti-IgG layer that occurs when just a few binding events are involved;
ii) the capturing of a whole IgG layer is still possible at much higher concentrations as no denaturation the anti-IgG layer occurs and so it can still bind about 10¹¹ IgGs.

To better characterize the invention disclosed in FIG. 4, two variants of Protocol B were implemented which are referred to as “Protocol C” and “Protocol D”. Specifically, “Protocol C” and “Protocol D” used a further buffer solution referred to as “HEPES buffer 3” (iₛ=5 mM, pH=7.4) and “HEPES buffer 4” (iₛ=150 mM, pH=6) for rinsing, respectively. In both cases, the incubation was always carried out in HEPES buffer 1. As in the case of Protocol B, the incubation with the antigens lasted 45 minutes and again involved 0.1 ml of solution, the rinsing lasted 20 minutes (again involving 3 ml of solution) and the restoring of the physiological conditions lasted 5 minutes (again involving 3 ml of solution).

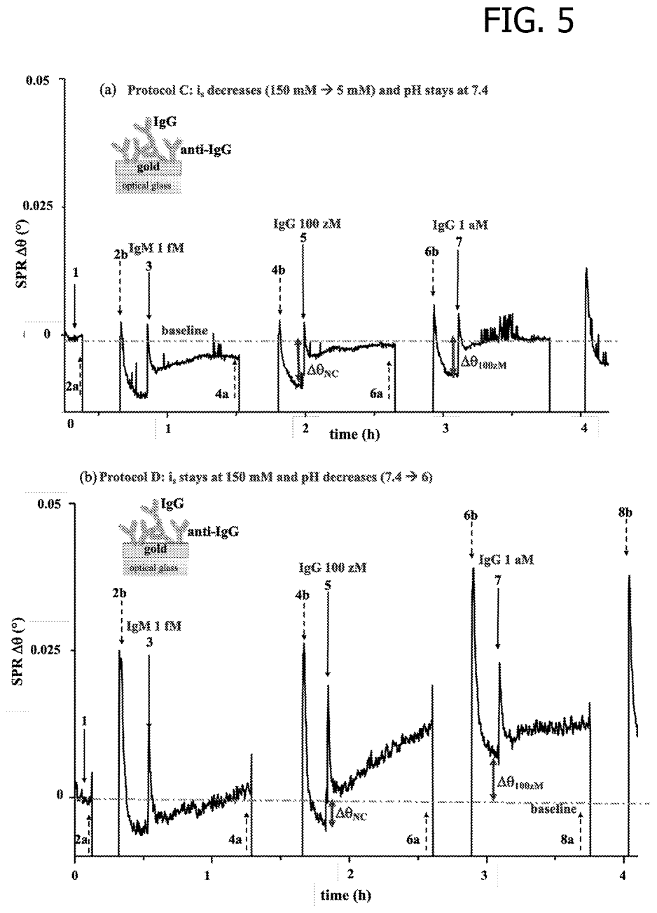

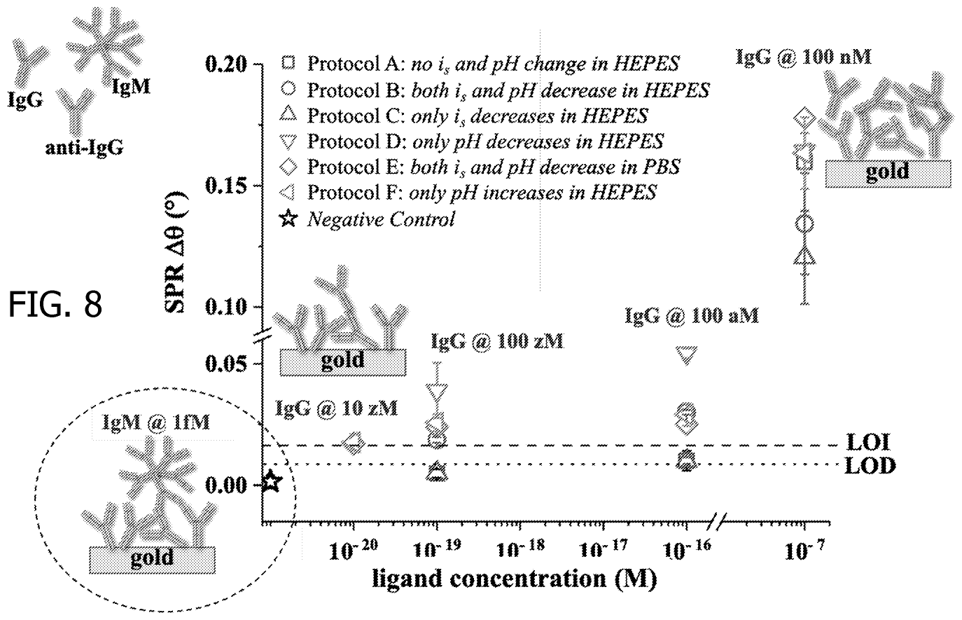

The data relevant to Protocols C and D are presented in FIG. 5(a) and FIG. 5(b), respectively where only steps 1-7 are shown for the sake of clarity. In both cases the injection of a 100 nM solution gave a large angle shift (data shown only in FIG. 8, vide infra). For FIG. 5(a) (Protocol C), no appreciable shift from the baseline is recorded (Δθ_{100zM}−Δθ_{NC}=0.003°±0.002° comparable to the instrumental error), until the IgG 100 nM solution is injected in the cell 3. As anticipated, here, a signal (not shown) comparable to FIG. 4(a)—step 14 is seen. The signal at 100 nM is seen also in the data gathered with Protocol D. Thus, proving also for Protocols C and D, no denaturation of the capturing layer occurs as well.

It can be concluded that the data presented in FIG. 5(a) show how the rinsing carried out in HEPES buffer 3 (involving an ionic strength lowering but no pH decrease as compared to the HEPES buffer 1), does not trigger an SPR Δθ shift as compared to the baseline until 100 nM IgG concentration is injected. This means that the sole change in the ionic strength is generates an effect alike to that seen when HEPES buffer 1 is used also as a rinsing solution.

A different scenario is seen with Protocol D, involving the rinsing with the HEPES buffer 4 where only the pH is lowered compared to HEPES buffer 1 (FIG. 5(b)). Here Δθ_{100zM}−Δθ_{NC}=0.037°±0.011° indicates that a quite large SPR signal shift is recorded compared to the baseline and to the negative control experiment (step 4a). In this case, the increasing Δθ shift starts as soon as the few IgGs are injected, showing that the effect of the rinsing at lower pH, is triggered by the exposure occurring before the affinity binding.

To assess the generality of the effect involved, a buffer and rinsing solutions different from HEPES have been tested. To this end, phosphate buffer saline (PBS at iₛ=163 mM and pH=7.4) solution was used for the antigen binding, while deionized water (iₛ=ca. 5 mM and pH=5.5 ca.) was used for the washing. This is referred to as “Protocol E”. Indeed, the PBS buffer includes disodium hydrogen phosphate, sodium chloride, potassium chloride, and potassium dihydrogen phosphate, so it is more complex than the HEPES-based buffer used so far. On the other hand, deionized water is not a buffer. However, this combination allows to change both the pH and the ionic strength more drastically. This more extreme protocol of binding and washing has been tested both on the physisorbed anti-IgG and on the nanostructured anti-IgG AuNPs-based capturing layers.

The gold-plated slide, covered with the pristine anti-IgG layer physisorbed from PBS or covered by the anti-IgG AuNPs, was allocated in the SPR cell 3. The negative control experiment involves, as for all the other protocols, the injection of the non-binding IgM antigen at 1 fM in the PBS solution incubated for 45 minutes. The sensor surface is then rinsed by injecting 0.1 ml of deionized water and left to rest for 20 minutes. Afterward, to restore physiological conditions, 0.1 ml of the PBS solution is injected and left to rest for 5 min. The experiment proceeds with the injection of IgG solutions in the PBS buffer 1 (0.1 ml) spanning the 10 zM-100 nM concentration range. Each incubation with the antigens lasts 45 minutes and is followed by a rinsing step in deionized water for 20 minutes and the re-establishment of the physiological conditions in PBS solution for 5 minutes.

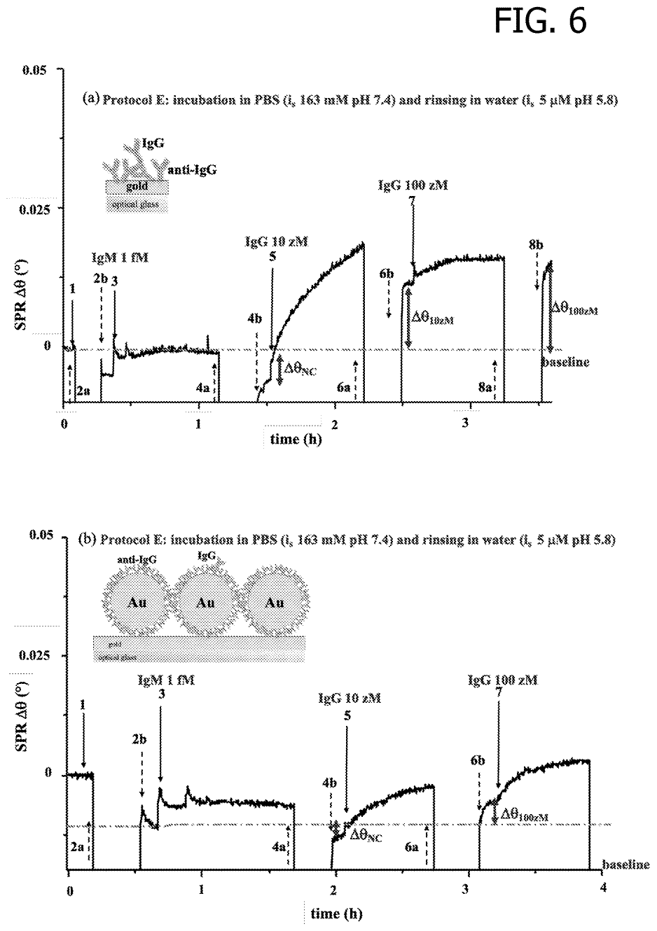

The SPR data taken according to Protocol E are shown in FIG. 6 and, in this case also, only steps 1-8 are shown. In analogy to previous Protocols, at 100 nM a large signal is measured (not shown). Each step of the protocol is marked by the relevant number and the baseline is the SPR signal level in PBS of the pristine anti-IgG layer. In FIG. 6(a) the sequence of steps is shown for the bare physisorbed anti-IgG layer while in FIG. 6(b) the same experiment is carried out on the anti-IgG layer physisorbed on gold nanoparticles that form a nanostructured film on the SPR slide.

In both graphs, after injecting the non-binding IgM (1 fM) a rather stable signal is recorded. Then washing with water and restoring in PBS the signal sets on a new level that falls below the baseline. When the 10 zM solution is injected a very relevant signal change is recorded that stabilizes after washing in water and restoring in PBS. A slight further slight change is seen at 100 zM. A remarkably high signal is recorded particularly on the bare physisorbed anti-IgG layer at 10 zM, namely when just one IgG is present in solution. Here a Δθ_{10zM}−Δθ_{NC}=0.0160±0.0040 is measured.

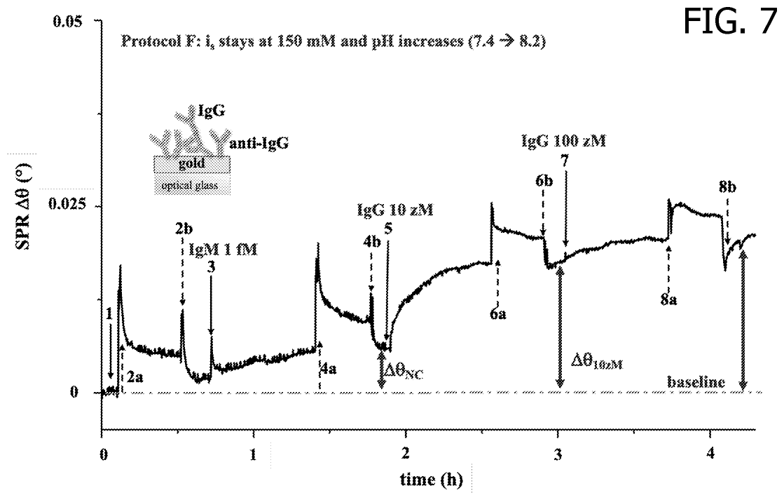

In “Protocol F” the pH was increased to 8.2 while the is was kept constant at 150 mM (“HEPES buffer 5”). As usual, the incubation was carried out in HEPES buffer 1. As in the case of Protocol B, the sensing with the antigens lasted 45 minutes, the rinsing 20 minutes, and the restoring of the physiological conditions lasted 5 minutes. The data relevant to Protocols F are presented in FIG. 7 where only steps 1-8 are shown and a rather large Δθ_{10zM}−Δθ_{NC}=0.0160±0.003° is measured in 10 zM. Also in this case the IgG 100 nM returns a signal (not shown) comparable to FIG. 4(a)—step 14.

The data relevant to all the experiments measured in Protocols A, B, C, D, E, and F are shown in FIG. 8. The Δθ signal for all the negative control experiments (17 replicates) is given as the average value (hollow star) while the error bar (falling within the symbol area) is one standard deviation. The LOD and the LOI level are indicated and all the data are averaged over at least 3 replicates and the error bar is taken as one standard deviation. The responses at the lowest IgG concentrations (10 zM, 100 zM, and 100 aM) are plotted on the left while those recorded at IgG 100 nM are on the right (note x and y-axis breaks). The data are displayed as shifts from the negative control experiments level. As it is apparent all the protocols invariably result in a large shift Δθ=0.153°±0.022° at 100 nM. This signal arises from the change in the dielectric function created by the building of the IgG layer on top of the anti-IgG. This is here addressed as the “IgG layer regime” and by means of de Feijter's equation^{28,21} an IgG coverage of (3.3±0.8)·10¹¹/cm² can be estimated at 100 nM that increases to (3.8±0.2)·10¹¹/cm² when the 440 nM IgG solution is assayed. The number of IgG molecules captured at these concentrations is comparable to that of the capturing anti-IgGs laying underneath.

At concentrations below 1 fM protocols B, D, E, and F result in an SPR Δθ shift beyond the LOI also at 10 zM, while protocols A and C return signals that are at the LOD or below. At these extremely low concentrations, no IgG layer can form, as at 10 zM only 1±1 IgG is present while 10±3 molecules are found in 0.1 ml at 100 zM (10⁴ at 100 aM). These few molecules are involved in the binding with an equal number of anti-IgG capturing antibodies. The conformational change associated with the binding of such a few IgG antigens entails a negligible electrostatic (dielectric function) change as an extremely low number of bindings involving a very minor portion of the 10¹¹ anti-IgGs forming the capturing layer 2, take place. Nonetheless a significant SPR Δθ shift is detected that is here addressed as the “single-molecule regime”. The signal recorded with Protocols B, E and F, and more markedly with Protocol D is to be ascribed to a change in the dielectric function occurring in the anti-IgG layer 2 that becomes detectable due to an amplification process.

### Role of the Buffer Ionic Strength and the pH Changes

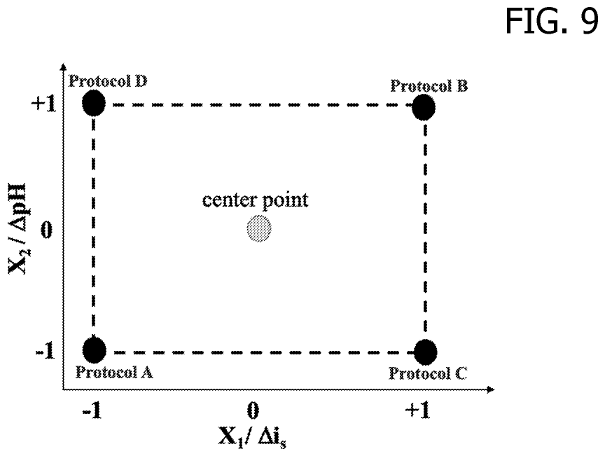

To better understand the nature of the invention that enables a reliable measure of a single antigen in 0.1 ml, an experimental design is set up according to the scheme given in FIG. 9. The study involves results from Protocols A, B, C, and D as those involve homologous HEPES-based buffers. The Δθ shifts measured, listed in Table 2, are estimated as the difference between the baseline value and the rinsing (Protocol A) or physiological conditions' restoring signal level (Protocol B, C, and D).

The experimental design is carried out according to a 2² full factorial approach to explore the domain, where Protocol A, Protocol B, Protocol C, and Protocol D represent the corners of the domain in which the SPR angle shifts Δθ have been measured. The 2² factorial design is based on two variables, namely the decrease of ionic strength (Δiₛ) and the decrease of the pH (ΔpH, entailing an increase in H+ concentration).

HEPES buffer 1 is always used in the incubation steps, while the other HEPES buffers 2, 3, 4 are used for the rinsing. Following a two-level design, the two quantitative variables have been encoded as X₁ and X₂, representing the Δiₛ and ΔpH respectively, settled at two levels, namely −1 (no variation) and +1 (full variation).

TABLE 2

| | | | | | | Δθ | | | Δθ |
|---|---|---|---|---|---|---|---|---|---|
| | | | | | | shift for IgG | | | shift for |
| # | | iₛ | | | | 100 | 100 | 100 | IgM |
| replicates | Protocol | (mM) | pH | X₁ | X₂ | zM | aM | nM | 1 fM |
| 1 | A | 150 | 7.4 | −1 | −1 | 0.003 | 0.006 | 0.160 | 0.002 |
| 2 | | 150 | 7.4 | −1 | −1 | 0.004 | 0.007 | 0.160 | 0.003 |
| 3 | | 150 | 7.4 | −1 | −1 | 0.005 | 0.013 | 0.160 | 0.003 |
| 4 | | 150 | 7.4 | −1 | −1 | 0.007 | 0.014 | 0.160 | 0.005 |
| 1 | B | 5 | 6.0 | +1 | +1 | 0.015 | 0.015 | 0.115 | −0.001 |
| 2 | | 5 | 6.0 | +1 | +1 | 0.013 | 0.011 | 0.147 | 0.003 |
| 3 | | 5 | 6.0 | +1 | +1 | 0.013 | 0.016 | 0.141 | 0.001 |
| 4 | | 5 | 6.0 | +1 | +1 | 0.011 | 0.012 | 0.092 | −0.007 |
| 5 | | 5 | 6.0 | +1 | +1 | 0.018 | 0.020 | 0.040 | −0.007 |
| 6 | | 5 | 6.0 | +1 | +1 | 0.018 | 0.021 | 0.080 | 0.002 |
| 7 | | 5 | 6.0 | +1 | +1 | 0.014 | 0.019 | 0.060 | −0.008 |
| 1 | C | 5 | 7.4 | +1 | −1 | 0.020 | 0.020 | 0.100 | 0.006 |
| 2 | | 5 | 7.4 | +1 | −1 | 0.010 | 0.010 | 0.160 | 0.009 |
| 3 | | 5 | 7.4 | +1 | −1 | 0.005 | 0.013 | 0.134 | 0.002 |
| 1 | D | 150 | 6.0 | −1 | +1 | 0.052 | 0.075 | 0.246 | 0.001 |
| 2 | | 150 | 6.0 | −1 | +1 | 0.034 | 0.053 | 0.143 | 0.001 |
| 3 | | 150 | 6.0 | −1 | +1 | 0.031 | 0.035 | 0.104 | 0.001 |

From a geometrical point of view, the 2² factorial design explores the corner of a square, meaning that, the model herein developed allows both variables to change simultaneously. The output of the experiment performed at the corners of the experimental domain, marked by the black circles (in FIG. 9), has been used to feed the model. In addition, two experiments have been performed at the center point, shown as a grey circle in FIG. 9, to assess the model's prediction capability.

The analysis based on multivariate linear regression provided the following model, correlating the SPR angle shift Δθ registered with sensing at 100 zM and the two coded variables as described in Equation 1 below:

$$SPR {Δθ}_{@ 100 zM} = 0.017 - 0.0 \; 05 {X}_{1}^{( * * )} + 0.0 \; 10 {X}_{2}^{( * * * )} - 0.0 \; 07 {X}_{1} {X}_{2}^{( * * * )} \qquad ( 1 )$$

where the significance level is indicated according to the usual convention: *=p<0.05, **=p<0.01, ***=p<0.001. Those p-values define the confidence interval of the Student's t-test used to evaluate the significance of each coefficient in the regression model.

The selected design, computed with 15 degrees of freedom, gives a maximum leverage of 0.33 in the whole experimental domain. The leverage, multiplied by the experimental variance, corresponds to the variance of the estimated response at that point. A leverage of 1 means that the model will predict the response with the same precision as the experiment, while a leverage <1 means that the response can be predicted with better precision than the experimental data collected under the same conditions. Hence, it is possible to infer that the model proposed holds an excellent predictive ability.

Moreover, the experimental value of the SPR angle shift Δθ registered at the center point is (0.017±0.005) degrees, which is not significantly different from the predicted value (0.017 degrees). Thus, the model is validated and accepted in the whole experimental domain.

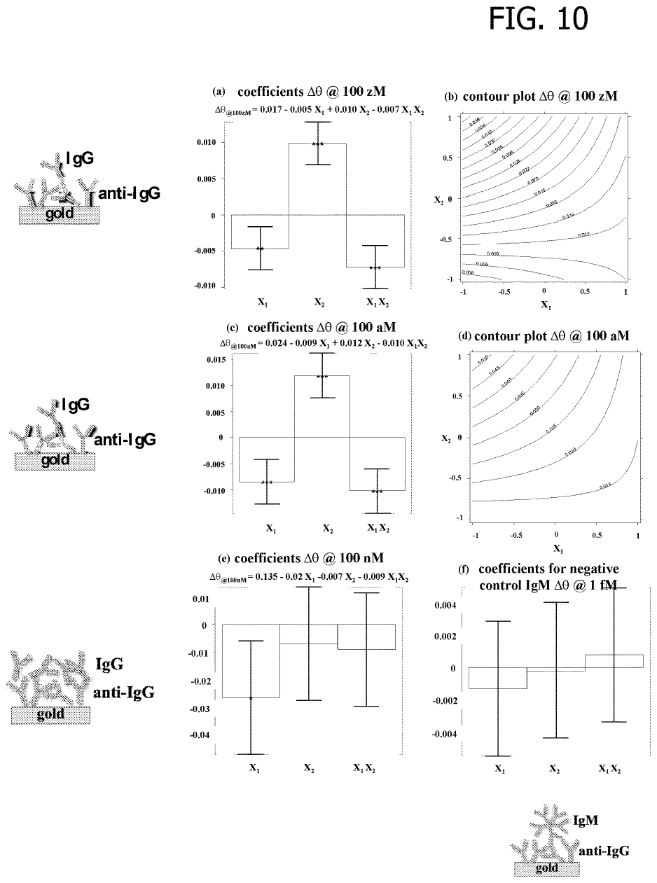

The coefficients of the model, pictured in FIG. 10(a), are all significant terms, although the linear terms of X₂ hold an absolute value larger than the other ones. In fact, reducing the pH of the rinsing buffer from 7.4 to 6, leads to an increase in SPR angle shift of one order of magnitude. On the other hand, a rinsing buffer with a lower ionic strength has a limited impact on the SPR angle shift. The coefficient for each term represents the change in the mean response associated with an increase of one coded unit in that term, while the other terms are kept constant. By changing the ionic strength of the rinsing buffer from 150 mM to 5 mM, the SPR angle shift variation below the LOD, while the pH change is as higher than the LOI, being hence significant.

The isoresponse contour plot, reported in FIG. 10(b), provides information about the interactions among the two variables X₁ and X₂. The geometrical shape of a linear model without interactions is a plane, leading to isoresponse lines that are parallel, while if relevant interactions are present, the contour plot shows a distorted plane with the isoresponse lines that are not parallel. This implies that the effect of a lower ionic strength of the rinsing buffer becomes more relevant only at higher pH differences. In comparison, at much lower pHs of the rinsing buffer, the ionic strength has little to no effect. Moreover, FIG. 10(b) confirms that the preferred (as well as providing the best results) condition to get an SPR response at very low IgG concentrations (in the explored experimental domain) corresponds to lowering the pH of the rinsing buffer.

On the other hand, a change in the ionic strength of the rinsing buffer is not significantly improving the SPR angle shift. This is in line with what has already been published concerning the effects of pH and Ionic strength on protein-protein interactions, unfolding, and aggregation for IgG antibodies.³⁴

For the SPR angle shift Δθ registered with sensing at 100 aM, the analysis based on multivariate linear regression provided the following model correlating and the two coded variables as in Equation 2 below:

$$SPR {Δθ}_{@ 10 \; 0 aM} = 0.024 - 0.0 \; 09 {X}_{1}^{( * * * )} + 0.0 \; 12 {X}_{2}^{( * * * )} - 0.0 \; 10 {X}_{1} {X}_{2}^{( * * * )} . \qquad ( 2 )$$

Equation 2 is line with the main features of the model computed for the sensing at 100 zM. The selected design, computed with 15 degrees of freedom, also produced a maximum leverage of 0.33. Moreover, the experimental value of SPR angle shift registered at the center point is (0.020±0.015) degrees, which is not significantly different from the predicted value (0.024). Thus, the model is validated and accepted in the whole experimental domain. The coefficient of the model, reported in FIG. 10(c), are all significant terms, and even in this case, the linear term of X₂ holds an absolute value larger than the other ones. The isoresponse contour plot, reported in FIG. 10(d), shows even in this case that the preferred (as well as providing the best results) condition to enhance the SPR angle shift in the explored experimental domain entails the lowering pH of the rinsing buffer, while reducing the ionic strength of the rinsing buffer does not produce an improvement in the SPR angle shift.

The analysis performed on the SPR angle shifts registered upon exposure to 100 nM of IgG returns the model given by Equation 3 below:

$$SPR {Δθ}_{@ 100 nM} = 0.135 - 0.02 {X}_{1}^{( * )} - 0.0 \; 07 {X}_{2} - 0.0 \; 09 {X}_{1} {X}_{2} . \qquad ( 3 )$$

FIG. 10 (e) shows that X₁, associated with the change in ionic strength, is the only significant coefficient in this case, implying that the dependence on the ionic strength of the rinsing buffer is the only aspect that matters. Also in this case, the experimental value of SPR angle shift Δθ registered at the center point is (0.118±0.040) degrees, which is not significantly different from the predicted value (0.135). Remarkably, in this case, reducing the ionic strength of the rinsing buffer from 150 mM to 5 mM produces a lowering of the SPR angle shift of about 15%, while the effect of the pH becomes negligible.

The very different models that are entailed at low concentrations (100 zM and 100 aM) and at 100 nM, fully account for the need to consider two completely different regimes in the SPR IgG assay at a capturing anti-IgG layer, namely: a “single-molecule regime” (10 zM-10 nM) where the change of the dielectric function involves an electrostatic rearrangement in the anti-IgG layer 2 and a “IgG layer regime” (>10 nM), where the dielectric function is modified by the building of a new layer on top of the capturing anti-IgG one. Finally, the SPR angle shifts registered during the negative control experiments are negligible, as it can be seen in FIG. 10(f), independently from the measuring protocol, namely, neither the ionic strength nor the pH changes of the rinsing buffer, matter.

Propagation Mechanisms that Enable the Single-Molecule Affinity Binding SPR Signal Amplification

The information gathered so far allows a deepening of the understanding of the propagation mechanisms that enable the signal amplification in the presence of a few single-molecule affinity binding events. Specifically, the following pieces of evidence, summarized in FIG. (8), can be recalled:
a washing of the detecting interface with a solution (HEPES-based buffer or deionized water) at lower or higher pH as compared to the physiological buffer (HEPES-based or PBS) where the binding occurs, is essential to enable the label-free optical detection of few affinity-binding events at a millimeter wide interface. A ΔpH from 7.4 to 5.5-6.0 or to 8.2 is a preferred option to serve this purpose.
the single-molecule regime in optical label-free assays according to the invention, necessarily entails a change in the dielectric function of the capturing layer comprising trillions of highly packed antibodies. Their density is 10⁴/μm², which is as high as that of proteins/receptors populating the surface of cells capable of single-molecule detection or tracking.^{35,36}
the bioelectronic single-molecule detections carried out with a transistor^{2,19-22} or with a Kelvin probe force microscopy,²³ entails the same ΔpH between the washing of the capturing layer and the binding carried out at physiological conditions. Hence, the washing step at lower or higher pH along with the single-molecule affinity binding at physiological conditions (Protocols B, D, E and F), rather than any applied electric or optical field, is at the basis of the single-molecule optical label-free regime according to the invention.
a lowering of the washing solution/buffer ionic strength (Δiₛ) does not enable, per se, the single-molecule regime. However, when associated with low pH changes it can help to increase the SPR signal toward IgG single-molecule detection.
The several reiterative exposures of the anti-IgG capturing layer to solutions that switch from physiological conditions (iₛ=150 mM, pH=7.4) to lower is and pH values or to higher pH values, do not result in the denaturation of these antibodies. Anti-IgGs are proven to capture IgGs which form a layer as dense as the anti-IgG one. Moreover, the number of IgG captured in protocols B, D, E, F is comparable to those deposited on anti-IgG in the experiments where no exposure to non-ideal physiological conditions occurs (see FIG. 2 and data relevant to Protocol A).

All these pieces of evidence can be used to get deeper insights into the amplification mechanisms that make the single affinity-binding event detectable at a large interface. So far, a domino-like spreading process has been evoked that originally entailed a self-assembled monolayer (a mixed SAM of activated-and-blocked 3-mercaptopropionic acid and 11-mercaptoundecanoic acid)¹⁹ to covalently attach the antibodies to the transistor Au-gate electrode. The hydrogen bonding network running through this chemical SAM was considered responsible for the cooperative interactions between the SAM chains.

It was supposed that a defect in the hydrogen bonding network would be created by the local electrostatic change generated by the single-affinity binding. Such a change eventually propagates thanks to the network fueled by the applied electric field associated with electronic detection.^{2,20}

Further data showed that the single-molecule detection was possible also when the layer of capturing antibodies was physisorbed^{21,23} at the gate electrode. Hence, the hydrogen bonding network associated with the SAM is not essential to the propagation. On the other hand, the data here shown proves that the gating field is not needed as well. Under these conditions to fully detail the actual propagation mechanisms an infrared spectroscopic investigation of the capturing layer at each step of protocols B, D, E, F is necessary to get information at the molecular level. On the other hand, based on the data published in the literature, some reasonable hypotheses on the nature of the propagation/amplification processes can be put forward. The main experiment results can be summarized as follows:
the capturing antibodies (or more in general the capturing specific binding pair-forming substance) layer shall be exposed to a pH change,
the layer that changes its dielectric properties comprises highly densely packed proteins,
no irreversible denaturation of the majority of the capturing antibodies (or more in general of the capturing specific binding pair-forming substance) occurs when exposed to non-physiological conditions.

These pieces of evidence point in the direction of one of the following mechanisms or a combination thereof:
The conformational spread connected with the phenomenon of allostery—the function of many proteins is regulated through allostery, by which a binding event at one site of a protein affects the behavior of another distant site. This long-range communication between protein sites represents the elementary process of signal transduction. Allostery is usually considered to be restrained within a single protein. However, the highly packed layer of intimately close antibodies can enable allosteric interactions between contiguous proteins. In fact, conformational changes have been shown also to propagate through extended systems of protein molecules in bacterial chemotaxis receptors, muscle ryanodine receptors, and actin filaments. The statistical mechanics of idealized linear and two-dimensional arrays of allosteric proteins have been shown, and in the analogous Ising models, arrays of closely packed units can show large-scale integrated behavior.³⁷
Proteins' aggregation (amyloid)—Proteins form aggregate and this process involves different stages: (i) partial monomer unfolding or misfolding; (ii) reversible self-association of folded or unfolded monomers; (iii) net irreversible aggregate formation via strong, stabilizing interprotein contacts such as hydrogen bonding and burial of hydrophobic amino acids—often this results in interprotein beta-sheet structures; (iv) subsequent aggregate growth via additional monomers; (v) growth via aggregate association to form larger soluble and/or insoluble aggregates. Stages (iii) to (v) are currently less well understood, while some aspects of aggregate growth and higher-order assembly have been studied for select systems, such as Alzheimer's antibodies, due to the roles of different types of antibodies aggregates in disease progression. Changes in protein-protein interactions, protein unfolding, and nonnative aggregation were assessed for a series of human IgG1 antibodies as a function of pH and solution ionic strength.^{34,38}
Resonance-assisted hydrogen bonds—Hydrogen bonds play vital roles in the formation of secondary structures of proteins, such as α-helix and β-sheet. A special property of these hydrogen bonds is their cooperativity, typified by the extra energy gain upon extension of the hydrogen bond units, (N—H . . . O═C)n. Several important protein behaviors, including the aggregation of β-amyloid peptides that may lead to Alzheimer's disease or mad-cow disease, are attributed to this cooperativity. The origin of cooperativity has been investigated mostly theoretically and a typical hypothesis is that resonance-assisted hydrogen bonds participate in the formation of protein secondary structures. Under this assumption, the lone pair electrons on the nitrogen atom and the n bond of the carbonyl group in peptide bonds would resonate to an enol-like structure.³⁹

Based on the information gathered from the literature, it could be conceived that a hydrogen bonding network is already present in the disordered deposit of physisorbed antibodies that connects the whole system. When physiological conditions are kept unchanged, the system is not triggered to change its dielectric function or its surface potential. In the presence of an affinity binding the conformational electrostatic change of the single anti-IgG involved does not propagate to other units. If the capturing layer 2 is exposed to solutions at different pH and ionic strengths but no affinity binding occurs, no significant change in the anti-IgG layer dielectric function is detected. Very surprisingly, when the capturing antibodies layer 2 is exposed to solutions at different pH (and ionic strengths though with a less critical impact) and an affinity-binding occurs, a sizable change in the anti-IgG layer dielectric function is detected. In this case, it can be supposed that the shift in pH and the presence of a local electrostatic change (dielectric rearrangements of dipoles due to the conformational change) generate the conditions for the spreading of the re-orientation of the dipole moments that probably lead to much more prominent interactions between the proteins maybe trough their α-helix and β-sheet. This induces phenomena such as the conformational spread, that bring the systems into a more stable configuration.

A generalization of this invention can be here called as many specific binding pair-forming substances—such as antigen/antibody couples—have been investigated so far with a bioelectronic device, among the others the following systems are noted (antibodies only): anti-Human Immunoglobulin G and anti-human Immunoglobulin M, anti-C-reactive protein, anti-HIV1 p24, anti-MUC1, anti-CD55, anti-S1 covid19, Purified-IgG against Xylella fastidiosa, anti-IL6 (monoclonal cytosine). Moreover, a single-molecule electronic response was proven also against streptavidin, avidin and neutravidin. Moreover, label-free, electronic single-molecule detection has been also demonstrated with peptides, DNA markers, such as KRAS, and microRNAs, such as miR-182-5p and SARS-CoV-2-encoded miRNAs modulation the virus replication.

Sensing a Single-Molecule at 10⁻²⁰ M with a Label-Free Optical Detection in a Simulated Real Setting.

To prove that protocols D, offering the highest response to a single binding event, can be used in a real (i.e., non-experimental) setting, the following proof-of-principle assay has been designed and carried out. A HEPES buffer 1 solution at iₛ=150 mM and pH=7.4 mimicking physiological conditions of the human plasma is added with the MERS-CoV spike S1 antigen at 1 fM concentration. This protein serves as an interferent that does not bind to the anti-p24 capturing layer. The MERS-CoV spike S1 HEPES buffer 1 is further added with HIV-1 p24 at 10 zM and 100 zM that binds to anti-p24 with high affinity. The HIV-1 p24 protein, selectively binding to the anti-p24 capturing antibody, is a marker for the HIV-1 infection whose early detection is highly relevant for the screening of asymptomatic patients. To this aim “Protocol D” is used for the assay according to the following steps:
0. Anti-p24 physisorption from PBS on a 0.4 cm² portion of the slide (2 hours);
1. Baseline (45 min): injection of 0.1 ml of the HEPES buffer 1 (iₛ=150 mM, pH=7.4);
2. a) Rinsing (20 min) injection of 3 ml of the HEPES buffer 4 (is =150 mM, pH=6);
   b) Restoring physiological conditions (5 min): injection of 3 ml of the HEPES buffer 1;
3. Negative control experiment with interferents (45 min): incubation with MERS-CoV spike S1 1 fM in HEPES buffer (injection of 0.1 ml) 1;
4. a) Rinsing (20 min): injection of 3 ml of the HEPES buffer 4;
   b) Restoring physiological conditions (5 min): injection of 3 ml of the HEPES buffer 1;
5. Sensing at 10 zM (45 min): incubation of MERS-CoV spike S1 1 fM with HIV-1 p24 10 zM in 0.1 ml of HEPES buffer 1;
6. a) Rinsing (20 min): injection of 3 ml of the HEPES buffer 4;
   b) Restoring physiological conditions (5 min): injection of 3 ml of the HEPES buffer 1;
7. Sensing at 100 zM (45 min): incubation of MERS-CoV spike S1 1 fM with HIV-1 p24 100 zM in 0.1 ml of HEPES buffer 1;
8. a) Rinsing (20 min): injection of 3 ml of the HEPES buffer 4;
   b) Restoring physiological conditions (5 min): injection of 3 ml of the HEPES buffer 1.

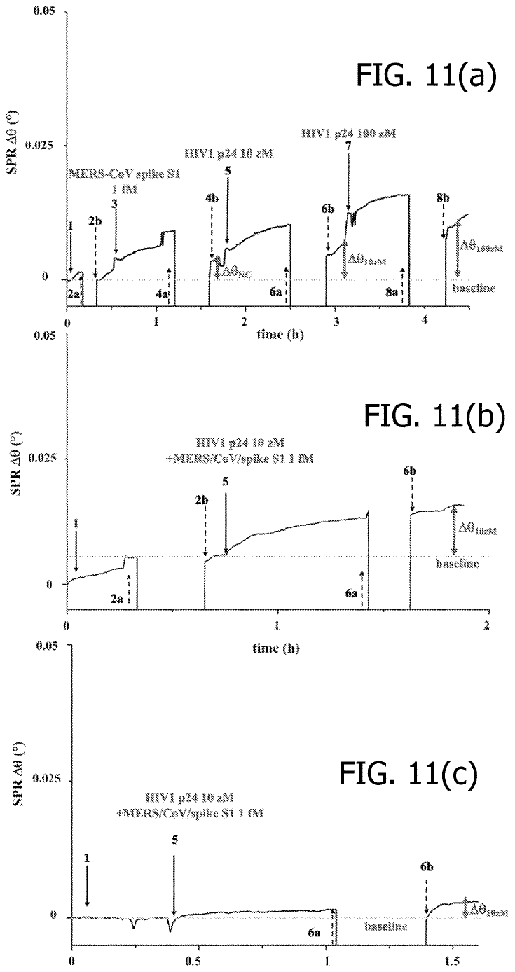

The results are shown in FIG. 11(a). As it can be seen the sensing is possible at the single-molecule level (10 zM) with and angle shift as high as Δθ_{10zM}−Δθ_{NC}=0.006°±0.001° and Δθ_{100zM}−Δθ_{NC}=0.010°±0.002°.

To better simulate a sensing in real condition the following protocol (modified protocol D) has been carried out. The method of the invention as performed in such conditions relies on an already deposited anti-P24 capturing layer, essentially indicating that in real (i.e., non-experimental) conditions an operator in a testing laboratory will be provided with a sensing kit including a cartridge delivered already functionalized, wherein the cartridge essentially comprises a substrate provided with a. So, the protocol becomes:
1. Baseline (45 min): injection of 0.1 ml of the HEPES buffer 1 (iₛ=150 mM, pH=7.4) this is addressed as the reference fluid;
2. a) Rinsing (20 min): injection of 3 ml of the HEPES buffer 4 (iₛ=150 mM, pH=6);
   b) Restoring physiological conditions (5 min): injection of 3 ml of the HEPES buffer 1;
5. Sensing at 10 zM (45 min): incubation of MERS-CoV spike S1 1 fM with HIV-1 p24 10 zM in 0.1 ml of HEPES buffer 1;
6. a) Rinsing (20 min): injection of the HEPES buffer 4;
   b) Restoring physiological conditions (5 min): injection of 3 ml of the HEPES buffer 1.

A net change of the signal respect to the baseline of Δθ_{10zM}=0.016°±0.002° can be seen already at 10 zM. This is a qualitative binary-type response (very much like to qualitative real-time PCR) with signal that is beyond the LOI level (0.015°). Such an assay allows to state, with a confidence level better than 99% (less than 1% of both false positives and false negatives), whether in a droplet of 0.1 ml at least one single targeted marker is present. It is also worth mentioning that: i) step 6 can be avoided (the signal is already consistently high enough at step 5; ii) steps 1, 2, and 5 can be much shorter as a stable signal, corresponding to 95% of the plateau, is recorded already after 20 minutes. So, the overall assay can last 40 minutes.

To assess whether the washing step should be only before the sensing or only after, the following protocol was measured:
1. Baseline (45 min): injection of the HEPES buffer 1 (iₛ=150 mM, pH=7.4) this is addressed as the reference fluid.
5. Sensing at 10 zM (45 min): incubation of MERS-CoV spike S1 1 fM with HIV-1 p24 10 zM in HEPES buffer 1;
6. a) Rinsing (20 min): injection of the HEPES buffer 4;
   b) Restoring physiological conditions (5 min): injection of the HEPES buffer 1;

In this case a very low change of the signal respect to the baseline of Δθ_{10zM}=0.003′±0.0020 can be measured at 10 zM. This proves that the single-molecule regime is not activated unless a washing step is performed before the injection of the affinity antigen.

To improve the binary sorting reliability, the use of a machine learning-based classifier can be implemented. To this end, a binary classifier can be designed, which is able to directly read raw data like those in FIG. 11 and produces an output attributing 0 to the negative samples (N) and 1 to the positive ones (P). The binary classifier can be trained with a training set comprising about 100 samples, divided among about 50 negative samples and 50 positives one. The prediction capability of the binary classifier can be evaluated using an external test set of about 40 samples assayed in blind. The binary classifier can be thus ad hoc designed to reliably differentiate among the negative samples from the positive ones with false positive and false negative errors below 1%.

Accordingly, in the light of the above, a method is defined according to embodiments of the invention for detection of binding events of specific binding pair-forming substances which can be outlined as comprising the following steps:
providing a layer of a first specific binding pair-forming substance on a substrate, said specific binding pair-forming substance having a density comprised between 10² and 10⁴ per μm² of said substrate
providing an enclosure of said layer of a first specific binding pair-forming substance with a first dielectric medium, the first dielectric medium comprising a first solution having a first pH value and a first ionic strength value,
rinsing the enclosure for a first period of time with a second dielectric medium comprising a second solution having a second pH value and a second ionic strength value, wherein at least said second pH value is different than said first pH value,
restoring said first dielectric medium having the first pH value and the first ionic strength value in said enclosure following said rinsing,
feeding a solution of a second specific binding pair-forming substance to the enclosure of said layer of a first specific binding pair-forming substance to cause an interaction between said first specific binding pair-forming substance and second specific binding pair-forming substance following said restoring the first dielectric medium in said enclosure, and incubating the enclosure for a second period of time, the interaction comprising a binding event when the first specific binding pair-forming substance and the second specific binding pair-forming substance provide said specific binding pair,
detecting a shift in a parameter representative of a dielectric function of the layer of a first specific binding pair-forming substance following said incubating the enclosure for a second period of time, the shift being defined as a variation of the parameter representative of a dielectric function of the layer of a first specific binding pair-forming substance from a first value occurring when said enclosure only includes said first dielectric medium following said restoring said first dielectric medium, and a second value occurring following incubating said enclosure for the second period of time,
comparing the shift Δθ with a threshold value (the limit-of-identification LOI, in particular), whereby when the shift exceeds the threshold value at least one binding event between said first specific binding pair-forming substance and second specific binding pair-forming substance has occurred.

As disclosed in the application, a preferred parameter representative of the dielectric function of the layer 2 of a first specific binding pair-forming substance is the SPR angle θ. However, as detection of the SPR angle θ and the related shift involves essentially optical techniques, other preferred embodiments may rely on a detection of a parameter representative of the dielectric function of the layer 2 through other optical-based techniques or optical-related parameters, in general without resorting to electrical inputs as it is, for instance, in a biological assay performed through a potentiometric measurement.

As apparent from the experimental protocols disclosed herein, as well as under the real (non-experimental) sensing conditions discussed in the foregoing, the rinsing may be carried out prior to and following the feeding a solution of a second specific binding pair-forming substance to the enclosure 4. In such case, detecting a shift in the parameter representative of a dielectric function of the layer 2 (SPR angle θ and shift Δθ) of a first specific binding pair-forming substance is carried out after the rinsing that follows the incubation of the enclosure 4 for the second period of time (i.e. following feeding of the solution of the second specific binding pair-forming substance to the enclosure 4).

In other embodiments, the rinsing is carried out only prior to feeding a solution of the second specific binding pair-forming substance to the enclosure 4, so that detecting the shift in the parameter representative of a dielectric function of the layer 2 of a first specific binding pair-forming substance is carried out following the incubation of the enclosure 4 for the second period of time.

### REFERENCES

1. Gooding, J. J. & Gaus, K. Single-Molecule Sensors: Challenges and Opportunities for Quantitative Analysis. Angew. Chemie—Int. Ed. 55, 11354-11366 (2016).
2. Macchia, E. et al. Large-Area Interfaces for Single-Molecule Label-free Bioelectronic Detection. Chem. Rev. (2022) doi:10.1021/acs.chemrev.lc00290.
3. De Angelis, F. et al. Breaking the diffusion limit with super-hydrophobic delivery of molecules to plasmonic nanofocusing SERS structures. Nat. Photonics 5, 682-687 (2011).
4. Macchia, E., Manoli, K., Di Franco, C., Scamarcio, G. & Torsi, L. New trends in single-molecule bioanalytical detection. Anal. Bioanal. Chem. 412, 5005-5014 (2020).
5. Wu, Y., Tilley, R. D. & Gooding, J. J. Challenges and Solutions in Developing Ultrasensitive Biosensors. J. Am. Chem. Soc. 141, 1162-1170 (2019).
6. Mayer, K. M., Hao, F., Lee, S., Nordlander, P. & Hafner, J. H. A single molecule immunoassay by localized surface plasmon resonance. Nanotechnology 21, 255503 (2010).
7. Anker, J. N. et al. Biosensing with plasmonic nanosensors. Nanosci. Technol. A Collect. Rev. from Nat. Journals 7, 308-319 (2009).
8. Zijlstra, P., Paulo, P. M. R. & Orrit, M. Optical detection of single non-absorbing molecules using the surface plasmon resonance of a gold nanorod. Nat. Nanotechnol. 7, 379-382 (2012).
9. Ding, S. Y. et al. Nanostructure-based plasmon-enhanced Raman spectroscopy for surface analysis of materials. Nat. Rev. Mater. 1, (2016).
10. Kretschmann, E. & Raether, H. Radiative Decay of Non Radiative Surface Plasmons Excited by Light. Zeitschrift fur Naturforsch.—Sect. A J. Phys. Sci. 23, 2135-2136 (1968).
11. Gedig, E. Surface Chemistry in SPR Technology. in Handbook of Surface Plasmon Resonance. (Royal Society of Chemistry, 2017). doi:10.1039/9781788010283.
12. Knoll, W. Interfaces and thin films as seen by bound electromagnetic waves. Annu. Rev. Phys. Chem. 49, 569-638 (1998).
13. Hifumi, E. et al. Elimination of ingredients effect to improve the detection of anti HIV-1 p24 antibody in human serum using SPR apparatus. Anal. Sci. 18, 863-867 (2002).
14. Malmqvist, M. Surface plasmon resonance for detection and measurement of antibody-antigen affinity and kinetics. Curr. Opin. Immunol. 5, 282-286 (1993).
15. Neumann, T., Johansson, M. L., Kambhampati, D. & Knoll, W. Surface-plasmon fluorescence spectroscopy. Adv. Funct. Mater. 12, 575-586 (2002).
16. Sarcina, L. et al. Surface Plasmon Resonance Assay for Label-Free and Selective Detection of HIV-1 p24 Protein. Biosensors 11, 180 (2021).
17. Vashist, S. K., Dixit, C. K., MacCraith, B. D. & O'Kennedy, R. Effect of antibody immobilization strategies on the analytical performance of a surface plasmon resonance-based immunoassay. Analyst 136, 4431-4436 (2011).
18. Nguyen, H. H., Park, J., Kang, S. & Kim, M. Surface plasmon resonance: A versatile technique for biosensor applications. Sensors (Switzerland) 15, 10481-10510 (2015).
19. Macchia, E. et al. Label-Free and Selective Single-Molecule Bioelectronic Sensing with a Millimeter-Wide Self-Assembled Monolayer of Anti-Immunoglobulins. Chem. Mater. 31, 6476-6483 (2019).
20. Macchia, E. et al. Single-molecule detection with a millimetre-sized transistor. Nat. Commun. 9, (2018).
21. Macchia, E. et al. A handheld intelligent single-molecule binary bioelectronic system for fast and reliable immunometric point-of-care testing. Sci. Adv. 8, 1-15 (2022).
22. Guo, K. et al. Rapid single-molecule detection of COVID-19 and MERS antigens via nanobody-functionalized organic electrochemical transistors. Nat. Biomed. Eng. 5, 666-677 (2021).
23. Di Franco, C. et al. Extended Work Function Shift of Large-Area Biofunctionalized Surfaces Triggered by a Few Single-Molecule Affinity Binding Events. Adv. Mater. Interfaces (2022) doi:10.1002/admi.202201829.
24. Macchia, E. et al. Why a Diffusing Single-Molecule can be Detected in Few Minutes by a Large Capturing Bioelectronic Interface. Adv. Sci. (2022) doi:10.1002/advs.202104381.
25. Singh, G. P. & Sardana, N. Smartphone-based Surface Plasmon Resonance Sensors: a Review. Plasmonics 17, 1869-1888 (2022).
26. Winspall 3.02 software. 3 http://www.res-tec.de/downloads.html.
27. Refractive Index reference value. https://refractiveindex.info/.
28. De Feijter, J. A., Benjamins, J. & Veer, F. A. Ellipsometry as a tool to study the adsorption behavior of synthetic and biopolymers at the air-water interface. Biopolymers 17, 1759-1772 (1978).
29. Liu, G. Y. & Amro, N. A. Positioning protein molecules on surfaces: A nanoengineering approach to supramolecular chemistry. Proc. Natl. Acad. Sci. U.S.A 99, 5165-5170 (2002).
30. Thompson, M., Ellison, S. L. R. & Wood, R. Harmonized guidelines for single-laboratory validation of methods of analysis (IUPAC Technical Report). Pure Appl. Chem. 74, 835-855 (2002).
31. Cowsill, B. J. et al. Interfacial structure of immobilized antibodies and perdeuterated HSA in model pregnancy tests measured with neutron reflectivity. Langmuir 30, 5880-5887 (2014).
32. Geerligs, I. E. J., Beijnen, J. H., Bekers, O., and U. & M., W. J. Quality control of protein and peptide drugs: Monoclonal antibodies and some biological response modifiers derived by recombinant DNA technology. Drug DeV. Ind. Pharm 19, 33-84 (1993).
33. Boswell, C. A. et al. Effects of charge on antibody tissue distribution and pharmacokinetics. Bioconjug. Chem. 21, 2153-2163 (2010).
34. Sahin Erinc, Grillo O. Adeola, Melissa D. Perkins, R. J. C. Comparative Effects of pH and Ionic Strength on Protein-Protein Interactions, Unfolding, and Aggregation for IgG1 Antibodies. J. Pharm. Sci. 99, 4830-4848 (2010).
35. Pichlo, M. et al. High density and ligand affinity confer ultrasensitive signal detection by a guanylyl cyclase chemoreceptor. J. Cell Biol. 206, 541-557 (2014).
36. Rieke, F. & Baylor, D. A. Single-photon detection by rod cells of the retina. Rev. Mod. Phys. 70, 1027-1036 (1998).
37. Duke, T. A. J., Le Novere, N. & Bray, D. Conformational spread in a ring of proteins: A stochastic approach to allostery. J. Mol. Biol. 308, 541-553 (2001).
38. Hartl, F. U., Bracher, A. & Hayer-Hartl, M. Molecular chaperones in protein folding and proteostasis.

### Nature 475, 324-332 (2011).

39. Zhou, Y. et al. Evidences for Cooperative Resonance-Assisted Hydrogen Bonds in Protein Secondary Structure Analogs. Sci. Rep. 6, 1-8 (2016).

1. A method for detection of at least one binding event of specific binding pair-forming substances, comprising:
providing a layer of a first specific binding pair-forming substance on a substrate, said first specific binding pair-forming substance having a density comprised between 10² and 10⁴ per μm² of said substrate,
providing an enclosure of said layer of the first specific binding pair-forming substance with a first dielectric medium, the first dielectric medium comprising a first solution having a first pH value and a first ionic strength value,
rinsing the enclosure for a first period of time with a second dielectric medium comprising a second solution having a second pH value and a second ionic strength value, wherein at least said second pH value is different than said first pH value,
restoring said first dielectric medium having the first pH value and the first ionic strength value in said enclosure following said rinsing,
feeding a solution of a second specific binding pair-forming substance to the enclosure of said layer of the first specific binding pair-forming substance to cause an interaction between said first specific binding pair-forming substance and second specific binding pair-forming substance following said restoring the first dielectric medium in said enclosure, and incubating the enclosure for a second period of time, wherein the interaction comprises a binding event of the at least one binding event when the first specific binding pair-forming substance and the second specific binding pair-forming substance provide said specific binding pair,
detecting a shift in a parameter representative of a dielectric function of the layer of the first specific binding pair-forming substance following said incubating the enclosure for a second period of time, the shift being defined as a variation of the parameter representative of the dielectric function of the layer of the first specific binding pair-forming substance from a first value occurring when said enclosure only includes said first dielectric medium following said restoring said first dielectric medium, and a second value occurring following incubating said enclosure for the second period of time, and
comparing the shift with a threshold value, whereby when the shift exceeds the threshold value, the at least one binding event between said first specific binding pair-forming substance and second specific binding pair-forming substance has occurred.

2. The method of claim 1, wherein said rinsing is carried out prior to and following said feeding the solution of the second specific binding pair-forming substance, and
wherein said detecting the shift in the parameter representative of the dielectric function of the layer of the first specific binding pair-forming substance is carried out after the rinsing that follows said incubating the enclosure for the second period of time.

3. The method of claim 1, wherein said rinsing is carried out only prior to said feeding the solution of the second specific binding pair-forming substance, and wherein said detecting the shift in the parameter representative of the dielectric function of the layer of the first specific binding pair-forming substance is carried out following said incubating the enclosure for the second period of time.

4. The method of claim 1, wherein said solution of the second specific binding pair-forming substance has a concentration in the range 10 zM-10 nM.

5. The method of claim 1, wherein said second ionic strength value is different from said first ionic strength value.

6. The method of claim 1, wherein said first specific binding pair forming substance comprises antibodies and said second specific binding pair forming substance comprises antigens, and wherein said binding event is a binding of said antigens to said antibodies.

7. The method of claim 1, wherein said parameter representative of the dielectric function of the layer of the first specific binding pair-forming substance is a Surface Plasmon Resonance (SPR) angle.

8. The method of claim 1, wherein said substrate is a metal substrate.

9. The method of claim 8, wherein said metal substrate comprises gold.

10. The method of claim 1, wherein said first solution has physiological pH and ionic strength values, including pH=7.4 and ionic strength=150 mM.

11. The method of claim 10, wherein said second solution has one of pH=6 or pH=8.2.

12. The method of claim 10, wherein said second solution has one of ionic strength=5 mM or ionic strength=5.5 μM.

13. The method of claim 1, wherein said first solution and said second solution are buffer solutions comprising 4-(2-hydroxyethyl)-1-piperazineethanesulfonic acid.

14. The method of claim 1, wherein said first solution and said second solution are buffer solutions comprising phosphate buffer saline (PBS).

15. (canceled)

16. The method of claim 7, wherein said threshold is a threshold SPR angle corresponding to a limit of identification (LOI).

17. The method of claim 16, wherein said threshold is 0.015°.
